(19) United States
(12) Patent Application Publication
(10) Pub. No.: US 2009/0270682 A1
(43) Pub. Date: Oct. 29, 2009

(54) SURGICAL INSTRUMENT

(75) Inventor: Hugo Matthieu Visser, Utrecht (NL)

Correspondence Address: PHILIPS INTELLECTUAL PROPERTY & STANDARDS, P. O. Box 3001, BRIARCLIFF MANOR, NY, 10510, US

(73) Assignee: Koninklijke Philips Electronics N.V., Eindhoven (NL)

(21) Appl. No.: 11/817,982

(22) PCT Filed: Mar. 10, 2006
(86) PCT No.: PCT/IB2006/050765

§ 371 Date: Sep. 7, 2007

(30) Foreign Application Priority Data
Mar. 14, 2005 (EP) 05101964.4

(51) Int. Cl. A61B 1/06

(52) U.S. Cl. 600/160

(57) ABSTRACT

The present invention relates to a surgical instrument. In particular the invention relates to a surgical instrument applicable in endoscopic surgery. In order to provide a simple and cheap solution for indicating the distance between a surgical instrument and its target, a surgical instrument (1) is suggested, which comprises a number of optical members (20, 21) to provide a diverging light emission in the direction of an instrument's target (5) in order to give an indication of the distance (10) between the instrument (1) and its target (5).

The present invention relates to a surgical instrument. In particular the invention relates to a surgical instrument applicable in endoscopic surgery.

Increasingly, medical interventions are being performed with so called “minimally invasive” methods. In such cases, the surgeon only makes small incisions through which instruments and an endoscope are brought inside a body. By using only small incisions the chance of infection, time to recover, and visibility of scars are reduced. The intervention, e.g. an operation or examination, is performed based on the endoscopic images viewed on a monitor.

One problem in such interventions is the absence of depth information. When using the endoscopic images on the monitor, the most important information the surgeon is missing is the distance from his surgical instrument to the “target”, for example a vein, a muscle, an organ wall etc. This makes all required manipulations a lot more time-consuming and risky.

From U.S. Pat. No. 4,281,931 an endoscope is known, which is adapted for measuring the distance to an object. The endoscope comprises a measuring apparatus, in which a laser beam is fed to a radial side of a rod lens by way of a light guide means. The light beam is scattered radially by the rod lens and reflected by a bowl-like mirror in the direction of the optical axis of the rod lens, finally bringing the light beam into a focus point on the object. The distance from the endoscope to the object is measured by calculating beforehand the relationship between the different positions from which the light beam can be fed to the rod lens and the corresponding distances. A disadvantage of this solution is its complex and expensive design. The distance has to be calculated, thus a direct indication of the distance is not possible. Moreover, in many cases the surgeon does not need to know the distance from endoscope to some ‘target’, but from the surgeon's instrument to the ‘target’.

It is an object of the present invention to provide a simple and cheap solution for indicating the distance between a surgical instrument and its target.

This object is achieved according to the invention by a surgical instrument comprising a number of optical members to provide a diverging light emission in the direction of an instrument's target in order to give an indication of the distance between the instrument and its target. This instrument is particularly applicable in endoscopic surgery.

The object of the present invention is also achieved by the use of a number of optical members as parts of a surgical instrument to provide a diverging light emission in the direction of an instrument's target in order to give an indication of the distance between the instrument and its target, particularly in endoscopic surgery.

A core idea of the invention is to utilize diverging light emission to determine the distance between two objects. For this purpose, no absolute measuring is carried out. Instead, relative measuring is used to give the operator, a surgeon or the like, an estimate of the distance to the target. For this purpose, the optical member adapted to emit the diverging light is positioned at the head of the surgical instrument.

Because no expensive technology has to be used, this solution is much cheaper than techniques known from the prior art. Only a simple diverging light emission is needed. The determination of distance is not performed using a measuring apparatus integrated into an endoscope. Instead, the invention is implemented using the surgical instruments employed during an intervention. Thus, the distance to the target can be determined for each single surgical instrument used.

The present invention might be used with a broad range of surgical instruments, for example with a surgical scalpel, scissors, knife, forceps, drill as well as with a surgical clamp, hemostat, retractor, trocar, perforator, or acus etc.

These and other aspects of the invention will be further elaborated on the basis of the following embodiments, which are defined in the dependent claims.

In a preferred embodiment of the present invention, the diverging light emission comprises at least two distinctive light beams. In this case the indication of the distance between the instrument and its target is given by the distance between the two light beams as they hit the target. In other words the operator can judge the distance from the instrument to the target from the separation of the beams as they are scattered from the target. The wider the beams are apart, the further the target is away. By using two separate light beams, each causing a light spot on the target, a very precise distance estimation can be performed.

In another embodiment of the invention, the diverging light emission comprises a light beam resulting in a projection of a light ring such that the indication of the distance between the instrument and its target is given by the dimension of the light ring. This embodiment is suitable especially for estimation of the distance to more complex structures with height differences. At points closer to the instrument, the diameter of the ring is less than at points further away. In fact, the ring is deformed into another closed curve. With this embodiment, relative distances can directly be estimated.

In a preferred embodiment, at least one of the number of optical members is coupled to a light source, preferably a laser. The use of laser light is especially advantageous because of the high brightness inherent to lasers.

In a further embodiment, it is proposed to guide the light to the instrument head using an optical fibre. Such fibres can be made as thin as 1 mm, allowing the laser source itself to remain outside the instrument. In other words the use of thin optical fibres allows the instrument to be comparatively small. This means that the necessary incisions can remain small. Because the optical fibre is thin, it can be very flexible, thus allowing the operator nearly unrestricted handling of the instrument.

The optical fibre preferably is a single-mode fiber. The advantage of using a single-mode fiber is that the light emerging from the fiber is very well defined. Moreover, single-mode fibers are the thinnest optical fibers.

In a further preferred embodiment of the present invention, the light source is part of the instrument. In this case no cable or fiber is needed at all, allowing a completely free handling of the surgical instruments. Such an internal light source might be especially advantageous in cases where the invention is implemented in a surgical instrument intended to remain in the body for a period of time without an operator using it, e.g. a surgical clamp.

In another preferred embodiment, at least one of the number of optical members is a lens adapted to collimate the light derived from the light source. Such a lens is used to collect the light in one direction, preferably to feed the optical member responsible for the diverging light emission.

The diverging light emission is preferably generated using a light guiding prismatic element. The prismatic form is designed to send the laser light off at one or more fixed angles. If the prismatic element is ring-shaped, cones of light beams diverge from the element at one or more fixed angles with respect to the ring's axis of rotation.

In still another embodiment of the invention, at least one of the number of optical members is adapted to provide a central light beam for targeting the instrument's target. If the prismatic element is adapted to provide the central light beam, no additional optical member is needed. Thus, the surgical instrument can be made small and lightweight.

Preferably, the surgical instrument is voice-controlled. A voice control unit is adapted to control the diverging light emission; the operator does not need to control the light emission and can handle the instrument like a traditional instrument. The voice control unit is preferably arranged outside the instrument in order to ensure a small and simple design of the instrument. However, parts of the voice control unit may also be integrated in the instrument itself.

These and other aspects of the invention will be described in detail hereinafter, by way of example, with reference to the following embodiments and the accompanying drawings, in which:

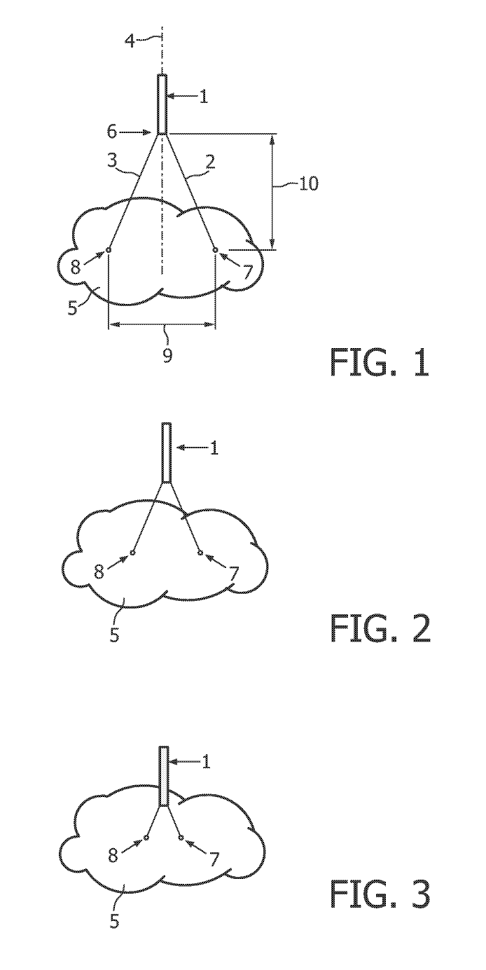

FIG. 1 is a schematic picture of a surgical instrument with two diverging laser beams in a first position,

FIG. 2 is a schematic picture of a surgical instrument with two diverging laser beams in a second position,

FIG. 3 is a schematic picture of a surgical instrument with two diverging laser beams in a third position,

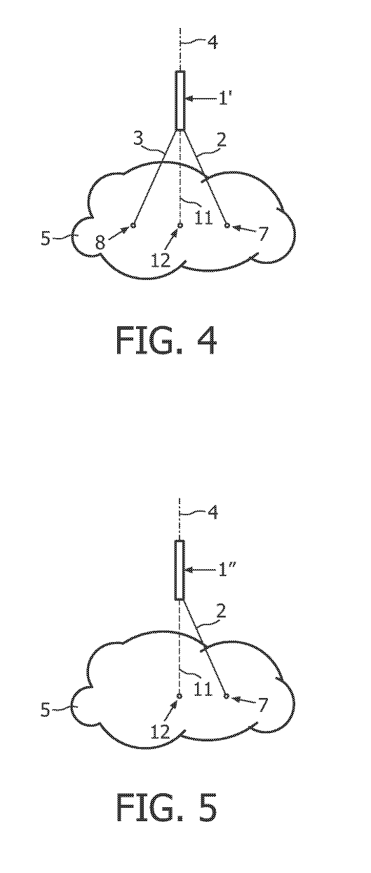

FIG. 4 is a schematic picture of a surgical instrument with two diverging laser beams and a central laser beam,

FIG. 5 is a schematic picture of a surgical instrument with a central laser beam and one additional laser beam for providing distance information,

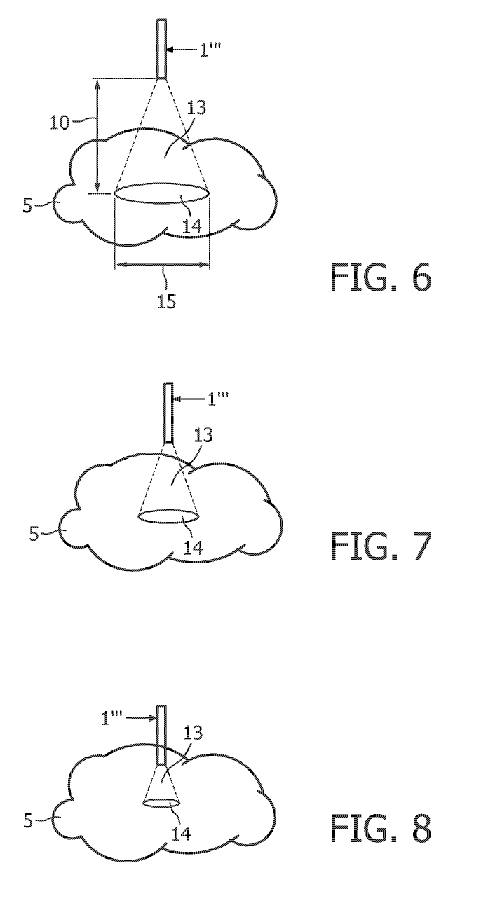

FIG. 6 is a schematic picture of a surgical instrument with a single diverging light beam in a first position,

FIG. 7 is a schematic picture of a surgical instrument with a single diverging light beam in a second position,

FIG. 8 is a schematic picture of a surgical instrument with a single diverging light beam in a third position,

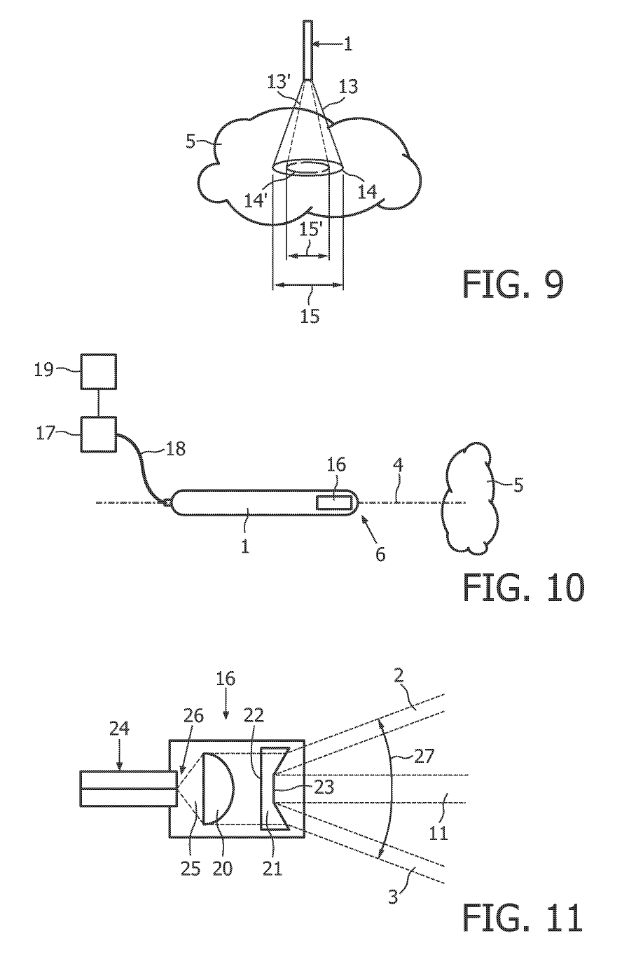

FIG. 9 is a schematic picture of a surgical instrument with multiple diverging light beams,

FIG. 10 is a schematic picture of a surgical instrument with an optical member,

FIG. 11 is a schematic picture of optical members within a surgical instrument.

In FIG. 1 a schematic picture of a surgical instrument 1 is shown with a diverging light emission in the form of two distinctive laser beams 2, 3. The centerline 4 of the instrument 1 indicates the direction in which the instrument 1 has to be moved in order to perform its function. For example, if the surgical instrument 1 is a scalpel, the centerline 4 indicates the direction in which the scalpel's blade is moved to the target 5, i.e. an organ wall or the like.

The two laser beams 2, 3 run from the head 6 of the instrument 1 in the direction of the instrument's target 5. Both laser beams 2, 3 run off the instrument's center line 4, generating two distinctive light spots 7, 8 on the target 5. The distance 9 between those spots 7, 8 gives an indication of the distance 10 between the instrument's head 6 and the target 5. In FIGS. 2 and 3, the surgical instrument 1 has been brought closer to the target 5. As a result, the light spots 7, 8 on the target 5 move closer together. As the light spots 7, 8 approach each other, the operator can judge from the distance between the spots 7, 8 how much space is left between the instrument 1 and the target 5.

In FIG. 4, a schematic picture of another embodiment of a surgical instrument 1′ is shown. Here the instrument 1′ is adapted to provide two diverging laser beams 2, 3 and a third central laser beam 11 running on the instrument's centerline 4. The central beam 11 is shown as a dashed line. It generates a third spot 12 on the target 5. Because the central laser beam 11 runs on the centerline 4 of the instrument 1′, it can be used for targeting.

FIG. 5 shows still another embodiment of a surgical instrument 1″. Again two distinctive laser beams are used for indicating the distance to the target 5. In this case, however, only one laser beam 2 running off the instrument's centerline 4 is used. As a reference for distance indication a central laser beam 11 is used. In other words, the operator may judge the distance between the instrument 1″ and the target 5 using the “fixed” center spot 12 and the “moving” second spot 7, which provides the distance information.

FIG. 6 shows a further embodiment of the invention. The surgical instrument 1′″ uses light beams 13 diverging at a single angle, resulting in a projection of a single light ring 14. As the instrument 1′″ moves in the direction of the target, the dimension, e.g. the diameter 15, of the ring projection 14 changes with the distance 10 to the target 5, as shown in FIGS. 7 and 8. In a further embodiment of the invention, as illustrated in FIG. 9, the surgical instrument 1′″ is modified in such a way that the diverging light beams 13, 13′ cause multiple rings 14, 14′ of different diameter 15, 15′, respectively. The advantage of such multiple rings 14, 14′ is that a larger area can be mapped in terms of height, or the same area can be mapped with more resolution. In another embodiment of the invention, (which is not illustrated in the Figures) the surgical instrument 1′″ is modified in such a way that the diverging light emission causes other geometrical projections, e.g. one or more squares etc. The advantage of rectangular projections is that a user directly gets an impression whether a target surface is convex or concave.

FIG. 10 shows a schematic picture of a surgical instrument 1 comprising an optical module 16 to provide a diverging light emission in the direction of an instrument's target 5. The optical module 16 is arranged at the instrument's head 6. It is coupled to an external light source 17 employing a connection cable 18. The connection cable 17 comprises an optical fiber (not shown) for guiding the light from the external light source 17 to the optical module 16. Since such optical fibers show a diameter of approximately 1 mm only, the connection cable 18 can be made very thin and flexible. For using the diverging light functionality of the instrument 1 no ON/OFF switch or the like is necessary, since the instrument 1 is voice-controlled. For this purpose, an external voice control unit 19 is connected to the external light source 17 to control the external light source 17.

FIG. 11 is a schematic picture of an optical module 16 within a surgical instrument 1. The optical module 16 is adapted to provide a diverging light emission according to FIG. 4. The optical module 16 shows a cylindrically symmetric layout and comprises a number of optical members, namely a lens 20 and a light guiding prismatic element 21. The prismatic element is ring-shaped and comprises a first optical surface 22 used as input surface and a second optical surface 23 used as output surface. An optical fibre 24, preferably a single-mode fiber with a fibre core diameter of approximately 1 to 10 microns, is used to guide the laser light from an external light source 17 to the lens 20. The emerging light cone 25 at the end 26 of the optical fibre 24 is collimated by the lens 20 and directed to the first optical surface 22 of the prismatic element 21. After passing the prismatic element 21, the light leaves the prismatic element 21 through the second optical surface 23. From the second optical surface 23 a central beam 11 and two further beams 2, 3 emerge at a predetermined fixed angle 27 of beam spread. All optical members 20, 21, 24 show dimensions between 1 and 2 mm, thus enabling the optical module 16 to be designed as a relatively small component.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit, may fulfil the functions of several means recited in the claims. Any reference signs in the claims shall not be construed as limiting the claim concerned.

### REFERENCE LIST

1 surgical instrument
2 laser beam
3 laser beam
4 centerline
5 target
6 instrument's head
7 light spot
8 light spot
9 spot distance
10 target distance
11 laser beam
12 light spot
13 light beam
14 light ring
15 diameter
16 optical module
17 external light source
18 connection cable
19 voice control unit
20 lens
21 prismatic element
22 first optical surface
23 second optical surface
24 optical fibre
25 light cone
26 fibre's end
27 angle of beam spread

1. A surgical instrument, particularly applicable in endoscopic surgery, comprising a number of optical members to provide a diverging light emission in the direction of an instrument's target in order to give an indication of the distance between the instrument and its target.

2. The surgical instrument as claimed in claim 1, wherein the diverging light emission comprises at least two distinctive light beams, such that the indication of the distance between the instrument and its target is given by the distance between the two light beams as they hit the target.

3. The surgical instrument as claimed in claim 1, wherein the diverging light emission comprises a light beam resulting in a projection of a light ring such that the indication of the distance between the instrument and its target is given by the dimension of the light ring.

4. The surgical instrument as claimed in claim 1, wherein at least one of the number of optical members is coupled to a light source.

5. The surgical instrument as claimed in claim 4, wherein the light source is adapted to emit laser light.

6. The surgical instrument as claimed in claim 4, comprising an optical fiber adapted to guide light from an external light source to at least one of the number of optical members.

7. The surgical instrument as claimed in claim 4, wherein at least one of the number of optical members is a lens adapted to collimate the light derived from the light source.

8. The surgical instrument as claimed in claim 1, wherein at least one of the number of optical members is a light guiding prismatic element.

9. The surgical instrument as claimed in claim 8, wherein the prismatic element is ring-shaped.

10. The surgical instrument as claimed in claim 1, wherein at least one of the number of optical members is adapted to provide a central light beam for targeting the instrument's target.

11. Use of a number of optical members as parts of a surgical instrument to provide a diverging light emission in the direction of an instrument's target in order to give an indication of the distance between the instrument and its target, particularly in endoscopic surgery.
